(19) United States
(12) Patent Application Publication
(10) Pub. No.: US 2011/0301459 A1
(43) Pub. Date: Dec. 8, 2011

(54) Surgical Procedure Bag

(76) Inventor: Morteza Gharib, Hollidaysburg, PA (US)

(21) Appl. No.: 13/103,167

(22) Filed: May 9, 2011

Related U.S. Application Data
(63) Continuation-in-part of application No. 12/794,761, filed on Jun. 6, 2010.

(51) Int. Cl. A61B 19/08; A61B 8/00

(52) U.S. Cl. 600/437; 128/849

(57) ABSTRACT

A sterile surgical procedure bag allows for rapid preparation of a surgical procedure while ensuring the sterility of the patient and the surgical instruments. The three dimensional shape of the bag creates a sterile space within which a surgical procedure may be performed. Sterile Instruments needed to perform the surgical procedure are secured to the interior of the bag in an organized and easily accessible way. The bag is attached to the surgical site. The bag further comprises openings for insertion of hands covered with sterile gloves into it, or the sterile instruments can be reached and manipulated through the bag's wall.

### CROSS-REFERENCE

This application is continuation in part of utility patent application No. 12/794,761 filed Jun. 6, 2010 and titled “A Surgical Procedure Bag”.

### STATEMENT REGARDING FEDERALLY SPONSORED RESEARCH

Not Applicable.

### FIELD OF INVENTION

This invention relates generally to apparatus used to create a sterile environment during surgical procedures, and more specifically to medical-grade coverings that eliminate the need for a conventional surgical drape and need for surgeon and his/her assistant(s) to wear a sterile gown while performing a surgical procedure.

### BACKGROUND

A surgical procedure is a procedure done on a patient or an animal. It may be a major procedure such as an intra-abdominal or intra-thoracic operation or a minor procedure such as the closure of a wound or the placement of a central line catheter or dialysis catheter. Alternatively, the procedure may be a simple advancement of a needle into the body for injection or aspiration or placement of various catheters within the body. A major risk in all types of surgical procedures is infection. In order to reduce the risk of infection, surgical procedures are to be performed under strict sterile conditions, which include the preparation of the surgical site with a disinfectant solution and the covering of the unsterile parts of the patient with sterile covering and the covering of the surgeon and the medical staff with various sterile and non-sterile coverings.

Typical coverings used by surgeons and medical staff include masks, hats, sterile gowns and drapes and sterile gloves. It may also include face guard or safety goggles if splashing of patient's bodily fluid to the medical staff's face is a concern. The donning of all of this apparel is not only time consuming, but there are situations when the surgeon is unable or unwilling to comply with all aseptic requirements. This may be due to a lack of time in emergency situations or, as happens with many minor surgical procedure or central line placement, due to lack of interest on the part of the surgeon in observing all sterility precautions. With the growing popularity of using ultrasound devices for central venous access procedures, the risk of contaminating a surgical site with an ultrasound probe also increases.

Moreover, as is currently done with minor surgical procedures and central line placements outside of hospital operating rooms, sterile packages containing the instruments to be used during the surgical procedure are opened and placed within reach of the surgeon, usually on a table next to the patient. It is not uncommon for the sterilized instruments to then touch unsterile objects or surfaces when they are removed from the packaging to the surgical site. Regardless of the type of procedure, the surgical site is also exposed to open air which may contain dust or different air born bacteria which may contaminate the surgical site. The medical staff, on the other hand, is exposed to contamination from surgical site and the patient's bodily fluids.

In the past several devices have been proposed that focus on the need to provide a barrier between the patient and the medical personnel. U.S. Pat. No. 5,316,541 describes a tent placed against a patient and a surgical procedure to be performed within that tent to protect the operating room from contamination. While this device is to be used in a standard operating room, it requires significant time consuming assembly, including addition of a surgical tray and a stand to the tent.

Therefore, there is a need in the art for an apparatus that isolates and separates the surgical site from surrounding area in any environment including a standard operating room setting as well as in emergency situations outside of an operating room. This is essential to ensure surgical instruments and surgical site will remain sterilized throughout an entire surgical procedure to reduce the risk of patient infection, while at the same time allowing a surgeon and his/her assistant to quickly prepare for and begin the surgical procedure.

### SUMMARY

The invention relates to an apparatus that ensures surgical instruments and surgical site will remain separated from surrounding environment and sterile throughout an entire surgical procedure, thereby reducing the risk of patient infection, while at the same time allowing a surgeon and his/her assistant to quickly prepare for and begin the surgical procedure. A bag is constructed of transparent, liquid impermeable, flexible, medical-grade material, such as polyurethane film or vinyl. The bag creates a sterile space inside it within which a surgical procedure may be performed. The bag may have any three dimensional shape or size conforming to the specific surgical procedure it is intended for.

The bag comprises at least a bottom surface and a top surface joining together to create an enclosed space within. The instruments needed to perform the surgical procedure are organized and secured inside the bag in an easily accessible and releasable manner. The bag is sterilized and packaged at the manufacturing facility. The bag's bottom surface is partially covered with a layer of medical grade adhesives from outside, such as an acrylic adhesive. The bottom surface will be positioned against the patient's body at the surgical site with the adhesive layer attaching to the patient's body and holding the bag in place.

The bag may have one or more openings for insertion of the surgeon's hands covered with sterile gloves, into it. In some embodiments the bag has a single opening for insertion of one hand, while in some other embodiments it may have two openings for insertion of two hands. In larger embodiments designed for more extensive procedures, it may even have any number of openings for multiple people reaching the instruments and performing or helping with the surgical procedure.

In certain embodiments the bag may have no openings for insertion of the hands and the surgeon may access and manipulate a surgical instrument through the wall of the bag rather than placing hands inside of it. In embodiments with one opening, one hand may be placed inside and the other hand helps from outside the bag. In embodiments which are designed to allow manipulation of a surgical instrument from outside of the bag, at least a portion of bag should be manufactured out of material thin and flexible enough to facilitate the grasping and manipulation of the instrument through the bag's wall.

Once the bag is placed over the surgical site, the surgeon may insert hands, covered with sterile gloves, into the bag to access sterile instruments and perform the procedure or in some embodiments a surgical instrument may be grasped and manipulated through the wall of the bag. After the surgical procedure is complete, the bag is removed from the patient, and the bag then is disposed of.

In one embodiment of the invention specifically customized to provide central venous access in order to place central venous catheters, hemodialysis catheters or pacemakers and so forth, a pouch for insertion of an ultrasound probe is added to the bag. This pouch will allow for the placement of the probe directly over the surgical site in order to visualize the tissue underlying the skin while at the same time preventing contamination of the surgical site by the probe.

### BRIEF DESCRIPTION OF DRAWINGS

FIG. 1 is a perspective view of a surgical bag according to an embodiment.

FIG. 2 is a perspective view of a surgical bag according to an embodiment.

FIG. 3 is a perspective view of a surgical bag according to an embodiment.

FIG. 4 is a perspective view of a surgical bag according to an embodiment.

FIG. 5 is a perspective view of a surgical bag according to an embodiment.

FIG. 6 is a perspective view of a surgical bag according to an embodiment.

FIG. 7a is a perspective view of a surgical bag according to an embodiment.

FIG. 7b is a perspective view of a surgical bag according to an embodiment.

### DETAILED DESCRIPTION

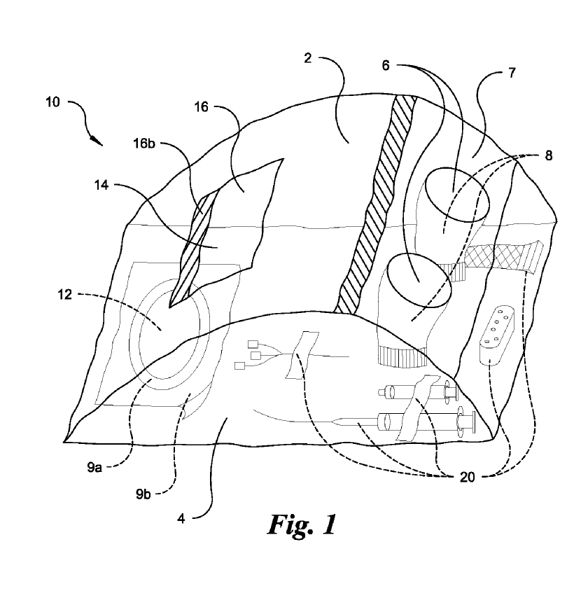

Referring to FIG. 1, a surgical bag 10 according to one embodiment of the present invention is shown. The bag 10 has sides, a bottom surface 4 and top surface 2. The bag 10 has a pair of openings 6 that allows for the insertion of a surgeon's hands into the bag 10. The openings 6 may engage and fit snugly around the surgeon's wrist. This engagement is meant to prevent back and forth movement of hands out and back into the sterile space. This is necessary to maintain sterility of the interior space during the procedure.

The openings 6 may be provided with sleeves 8. The sleeves 8 are sealed to the edges of the openings 6 and placed inside the bag 10. The sleeves 8 will cover the surgeon's wrist, and forearm, and provides flexibility of surgeon's hand movement inside the bag 10 while maintaining the sterility of the bag's 10 interior. The sleeve 8 may be constructed out of an elastic and stretchable material. The openings 6 may also have a protective cover 7 that covers the opening 6 from outside of the bag 10.

The protective cover 7, if provided, needs to be released from the bag 10 and removed from the openings 6 before the surgeon inserts his/her hand(s) into the openings 6. The bottom surface 4 may have opening 12. The opening 12 may be sized to fit over the surgical site of the patient and to allow the surgeon to access the patient's body so that the surgical procedure may be performed. The opening 12 may be provided with a layer of medical grade adhesive 9a on periphery of its exterior surface. The adhesive 9a will attach to the patient's body, at the surgical site, and maintain the bag 10 in place. The opening 12 may be provided with protective cover 9b. Cover 9b will cover the opening 12 from outside. Cover 9b, if provided, needs to be removed before the bag 10 is positioned against the surgical site.

Alternatively cover 9b may be releasable from inside the bag 10 after the bag is positioned over the surgical site. In certain embodiments the bottom surface 4 does not have any opening. In these embodiments, underneath of bottom 4 at least partially is covered with a layer of adhesive. An opening will be created after the bag 10 is placed over the surgical site by cutting and removing portions of the bottom surface 4 by the surgeon or a procedure may be performed through bottom surface 4.

In certain embodiments of the invention, the bag 10 may also comprise an access port 14. The access port 14 is an opening on the bag 10 that provides access to the interior of the bag. The access port 14 may be covered with a lid 16. The lid 16 may be resealable against the bag 10. The lid 16 may comprise tab 16b. The tab 16b can be used by an assistant to lift the lid 16. By lifting the lid 16, an assistant can insert additional sterile items into the interior of the bag 10 as needed by the surgeon. Used items can be removed from the bag 10 through the access port 14 as well. Care must be taken not to contaminate the interior of the bag 10 when adding items thereto or removing items therefrom through the access port 14. The access port 14 and lid 16 should be constructed in such a way as not to obstruct viewing of the interior of the bag 10.

One or multiple sterile instruments 20 are placed inside the bag 10. The instruments 20 are organized in an easily accessible way and fastened to the interior of the bag 10 in a releasable manner. The type of instruments 20 placed inside the bag 10 depends on the type of surgical procedure the bag 10 has been customized to accommodate. The instruments 20 can include needles, syringes, central line catheters, hemodialysis catheters, guide wires, regional anesthesia needles and catheters, scalpels, sharp containers, gauzes, surgical sutures, alcohol preps and other disinfectants, packs of conductive gels, bottles of local anesthetics, or other medications or any other items needed to perform the particular surgical procedure. In an alternate embodiment, the sterile instruments 20 may be added to the bag 10 at the patient bedside. The bag 10 at least partially is made of any transparent medical grade material or plastic that allows the interior of the bag 10 to be easily visible.

After the surgical site is prepped in the usual manner, the covering 9b, if provided, is removed and the bag 10 is positioned on top of the surgical site. The cover 7, if provided, is then removed and the surgeon's hands, covered with sterile gloves, are placed inside the bag 10 through the openings 6, and the surgical procedure performed in a sterile environment. As can be seen in FIGS. 1, because the surgical environment created inside the bag 10 is separated from outside exposure, there should be no need for covering of the rest of the patient's body with sterile drape or the surgeon and the medical staff to wear sterile gowns.

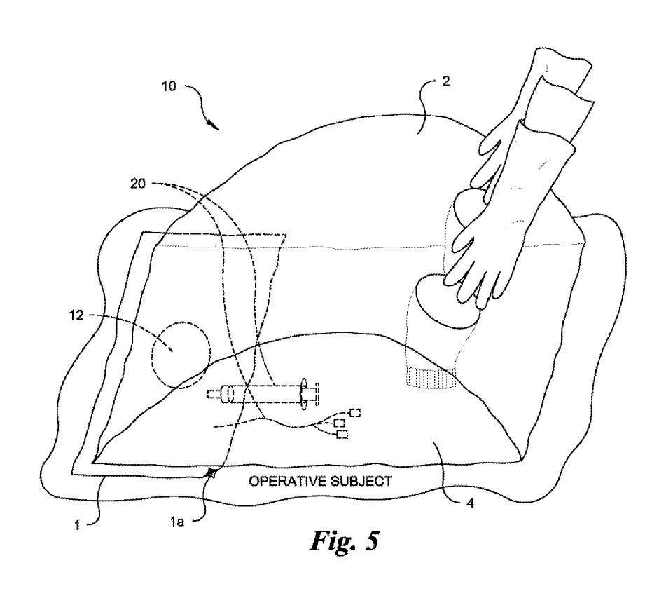

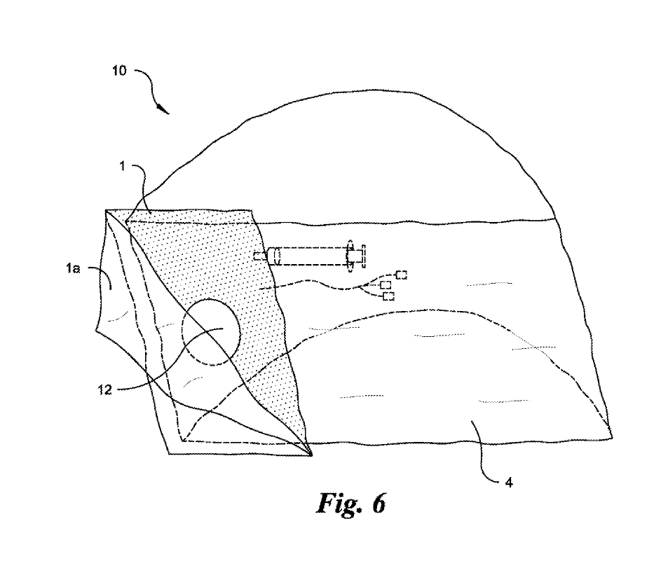

Referring to FIGS. 5 and 6, the bag 10 may further comprise a layer 1. Layer 1, is partially affixed to the underneath of the bag 10. In embodiments which have opening 12, the layer 1, is affixed to the periphery of opening 12. Layer 1 is covered with a layer of adhesive on the underneath side and further covered with removable backing 1a. Backing 1a may also cover opening 12 from outside. Upon removing backing 1a, the layer 1 is placed and attached to the operative site. Layer 1, is a soft and flexible layer that will follow surgical site's shape and will hold the bag 10 in place firmly and allows for greater flexibility of bag 10 around the operative site.

The present invention has important implications and uses for surgical procedures done in emergency situations or outside of operating rooms, such as surgical procedures done in military zones and so forth, where dust from the environment may prevent a sterile procedure from being performed. Even in controlled hospital environments, the isolation of the surgical site from surrounding environment provided by the present invention will make all medical staff present for the procedure compliant with sterility requirements. Because all the instruments 20 needed for the surgical procedure are organized and accessible inside the bag 10, the surgical procedure can often be done much faster and the risk of accidental contamination of the surgical site and the instruments 20 is significantly reduced. Simultaneously, because the surgical site is isolated, the medical staff is also protected from contamination from possible splashing droplets of patient's blood and bodily fluid.

The size and the shape of the bag 10 may be customized to suit the surgical procedure intended to be accomplished. The bag 10 can be customized for different surgical procedures and can have multiple openings 6 for the insertion of assistants' hands and additional openings for insertion of other equipment including electric wiring or different tubing for carrying different fluids. The bag 10 at least partially should be made of transparent medical-grade material. Although the bag can be made out of material with different rigidity, making it out of flexible material has the advantage of having more flexibility for hand movement inside the bag 10.

Being soft and collapsible also makes packaging and storing the bag 10 simpler. The bottom surface 4 may be soft and flexible to follow the contour of the patient's body. The bag 10 or at least the bottom 4 should be impermeable to liquids to prevent patient's body fluids from leaking into the bag 10 and contaminating the sterile environment. Interior surface of the bottom 4 may also be comprised of absorbent material to absorb any blood or bodily fluid that may be present as the result of surgical procedure.

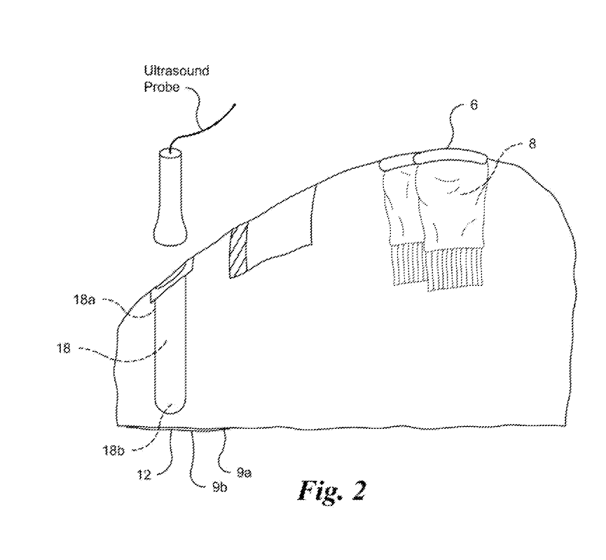

In one embodiment shown in FIG. 2, the bag 10 is comprised of a pouch 18. The pouch 18 is shaped to allow the insertion of an ultrasound probe. The pouch 18 can be made of any thin medical-grade material or plastics that is impermeable to liquids, which allows for easy penetration of an ultrasound signal to and from the ultrasound probe. The pouch 18 is sized to accept a plurality of commercially-available ultrasound probes. The pouch 18 has an open end 18a and a closed end 18b and is attached to the bag 10 at the open end 18a.

The patient's skin over the target vessel or nerve is prepped and the bag 10 is positioned over the target vessel, nerve or any other target tissue. The ultrasound probe is then placed inside the pouch 18 and the surgeon places his/her hands, covered with sterile gloves, through the openings 6 and inside the bag 10. Conductive gel may be placed inside the pouch 18 prior to probe insertion. The surgeon can hold the ultrasound probe from inside the bag 10 to visualize the target vessel or nerve or other underlying tissues without contaminating the surgical field. Alternatively, a bag without a pouch can be used for procedures which require ultrasound visualization. In the absence of a pouch, an assistant may be needed to place and hold the ultrasound probe over the target tissue.

In certain embodiments, the pouch 18 can hang free underneath the top surface 2 with the closed end 18b freely moving inside the bag 10. Alternatively, the pouch 18 may be anchored to the bottom surface 4. This anchoring is intended to maintain the head of the ultrasound probe over the skin over the target tissue. This anchoring may be rigid, semi-rigid, or even may include flexible anchors or joints to allow the ultrasound probe position and the angle against the patient's skin to be adjustable.

In certain embodiments, the bag 10 may further have at least one additional opening for permitting access into the interior space of the bag 10, for electric wiring or tubing for carrying different fluids. This embodiment particularly may be used for quickly creating a sterile environment and to prepare for placement of a regional anesthesia catheter. The opening may have means for engaging the electric wiring and fluid tube. This engagement is meant to prevent back and forth movement of wiring and the tube out and back into the sterile space.

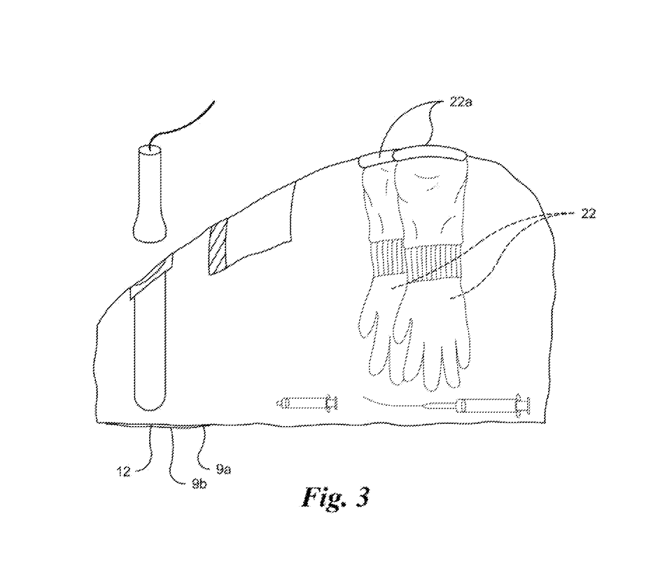

In one embodiment shown in FIG. 3, the surgical gloves 22 are affixed to the bag and positioned inside the bag 10. The proximal ends 22a of the surgical gloves 22 are sealed at the edges of the openings 6. This embodiment can be made with different glove sizes for different hand sizes. One advantage of having surgical gloves 22 incorporated into the bag 10 is that the surgeon will spend less time in preparation and can frequently remove his/her hands out and put back in the bag 10 without contaminating the interior of the bag 10.

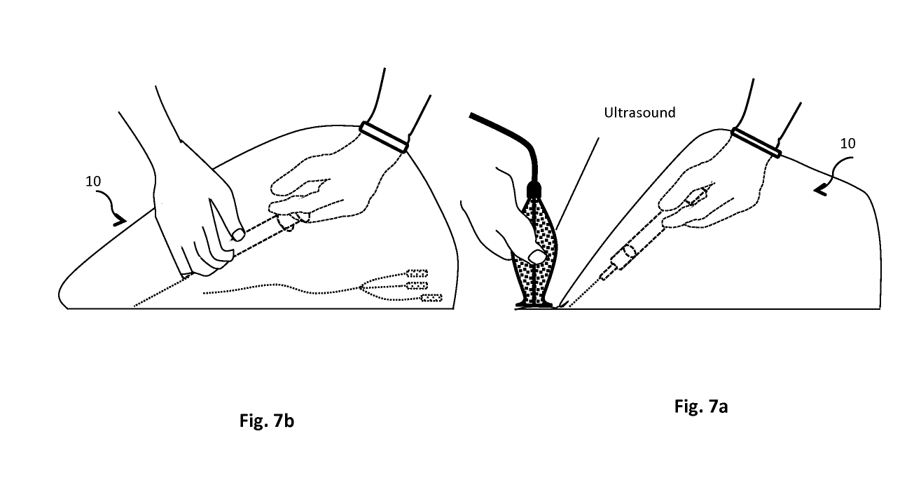

In one alternate embodiment shown in FIGS. 7a and 7b, the procedure bag 10 is shown having only one opening 6 for insertion of a hand. In this embodiment, one hand is covered with a sterile glove and placed inside bag 10, while the other hand is free outside the bag. The surgeon then may use both hands to manipulate the surgical instrument inside the bag, one hand from inside the bag and the other one from outside. In this embodiment the bag 10 at least partially should be constructed of thin material with enough flexibility that allows grasping and manipulation of instruments through its wall by the outside hand. Using this embodiment will allow the surgeon to have a free hand outside of the bag to operate any other instrument he/she may need, including holding and manipulating an ultrasound probe over and through the bag 10.

In certain embodiment the bottom 4 may be separable from the rest of the bag, so at some time during the procedure, when more dexterity is needed, the rest of the bag may be removed and surgical procedure may continue using the bottom layer as a regular surgical drape. This separation may be desirable for example in the embodiments with one opening for insertion of a single hand. For example when a central line has been placed using one hand inside and the other hand outside of the bag 10, then the bag 10 minus the bottom 4 is removed, the surgeon then may use bottom 4 as a regular drape and use both hands, covered with sterile gloves, to place surgical sutures around central line catheter.

In some embodiment both hands covered with sterile gloves may be placed in one opening 6. In this embodiment the opening 6 should be large enough to allow insertion of both hands into bag 10. In yet another embodiment the top layer 2 may be made without opening 6. In this particular embodiment, items 20 are manipulated through walls of the bag 10. In certain embodiment additional openings for the insertion of one or more assistants' hands may be provided.

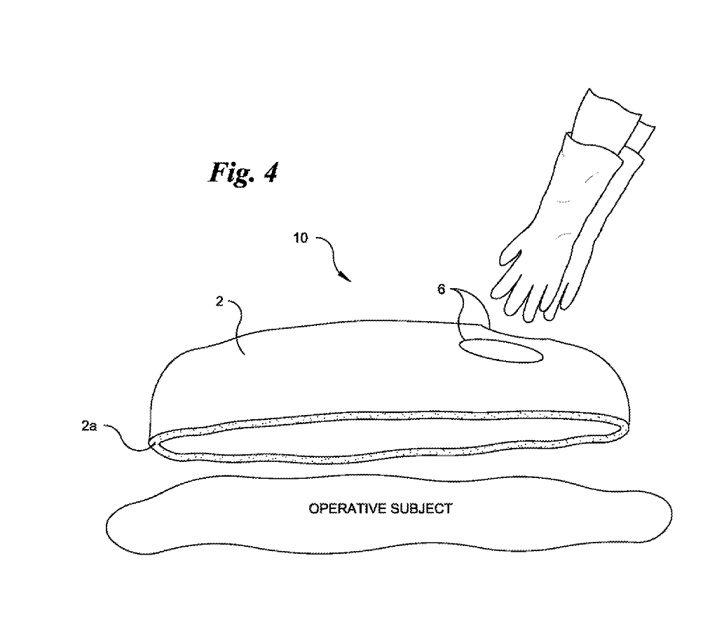

In an alternative embodiment shown in FIG. 4 the bag 10 is comprised of only top 2. Upon placement of top 2 over a subject, a three dimensional space is created by the subject body from underneath and top 2 from above. Top 2 may have a layer of adhesive 2a covering the edge of the top 2 and a removable backing may cover adhesive 2a. The adhesive layer 2a can maintain top 2 firmly at the surgical site. Similar to other embodiments, this embodiment can have one or multiple openings for insertion of hands or it may be constructed with no opening. In this embodiment the invention more resembles a regular surgical drape but the surgical procedure is performed underneath it, as opposed to a regular surgical drape which the procedure is performed above it.

In smaller embodiments customized for minor operations or central vascular access and regional anesthesia catheter placement, the bag 10 may be made of a material that, while soft and flexible, allows the bag 10 to naturally maintain its three-dimensional shape to create the space necessary for free movement of the surgeon's hands inside the bag 10. Placement of the hands inside the bag may help to maintain this shape. The bag may partially be constructed of more rigid but still flexible material to allow the bag 10 to stay upright. In larger bags, or bags made with very thin walls, means to prevent the bag 10 from collapsing may be needed. These means may include a variety of lightweight skeletons, as it is known in prior art, to be placed inside or outside of the bag 10. The skeleton may be flexible or collapsible to allow easy packaging and storage

I claim:

1. A surgical procedure bag at least partially constructed of transparent material and having sides, a top surface and a bottom surface, defining a sterile interior space, said bag comprising a means for reaching and manipulating a sterile surgical instrument housed within said space.

2. The surgical procedure bag of claim 1, wherein said means comprises at least one opening in said bag adapted for insertion of a hand into said bag.

3. The surgical procedure bag of claim 2, wherein said opening comprises a means for engaging said hand to prevent moving said hand out and back into said sterile space.

4. The surgical procedure bag of claim 2, wherein said bag further comprises a sleeve attached to said opening.

5. The surgical procedure bag of claim 2, wherein said bag further comprises a sterile surgical glove affixed to said opening.

6. The surgical procedure bag of claim 1, wherein said means for reaching and manipulating a sterile instrument housed within said space comprises at least a portion of said bag being constructed of thin flexible material that allows a user to reach into said space through said bag.

7. The surgical procedure bag of claim 1, wherein said bottom surface is adapted to be placed over a surgical site.

8. The surgical procedure bag of claim 1, wherein said bag further comprises a means for removably adhering to a surgical site.

9. The surgical procedure bag of claim 8, wherein said means comprises a flexible layer partially affixed to said bottom surface, wherein said flexible layer is covered with medical grade adhesive and further covered with a removable backing.

10. The surgical procedure bag of claim 1, wherein said bag further comprises a pouch for insertion of an ultrasound probe.

11. The surgical procedure bag of claim 1, wherein said bag further comprises an access port.

12. The surgical procedure bag of claim 11, wherein said access port further comprises a flap which covers and seals said port.

13. The surgical procedure bag of claim 1, wherein said bag further comprises at least one opening for permitting access to said sterile interior space for electric wiring and tubing for carrying fluids.

14. The surgical procedure bag of claim 1, wherein said bag further comprises at least one sterile surgical instrument placed inside said bag.

15. The surgical procedure bag of claim 14, wherein said instrument is releasably secured to the interior of said bag.

16. The surgical procedure bag of claim 1, wherein said bag is at least partially constructed of material having enough rigidity to maintain a three dimensional shape of said bag.

17. The surgical procedure bag of claim 1, wherein said bottom surface is separable from the rest of said bag.

18. A surgical procedure bag comprising a top surface and a bottom surface, said surfaces joined together to define a sterile interior space, said bag having at least one sterile surgical instrument housed within said space, said bag further comprising a first means for removably attaching said bag to a surgical site and a second means for reaching into said bag and manipulating said instrument to perform a surgical procedure.

19. A method of quickly creating a sterile environment for performing a surgical procedure, said method comprising steps of:
a) providing a sterile transparent bag, said bag comprising an internal, three-dimensional space and a means for reaching and manipulating a surgical instrument housed within said space;
b) placing at least one sterile surgical instrument inside said space;
c) placing said bag over a surgical site; and
d) manipulating said instrument through said bag and performing the surgical procedure using said instrument.

20. The method of claim 19, wherein said method further comprises steps of:
a) affixing a pouch to said bag;
b) inserting an ultrasound probe into said pouch; and
c) visualizing underlying body tissues using said ultrasound probe.
